(19) United States
(12) Patent Application Publication
(10) Pub. No.: US 2025/0090339 A1
(43) Pub. Date: Mar. 20, 2025

(54) GLENOID COMPONENT

(71) Applicants: Smith & Nephew, Inc., Memphis, TN (US); Smith & Nephew Orthopaedics AG, Zug (CH); Smith & Nephew Asia Pacific PTE. Limited, Singapore (SG)

(72) Inventors: Charles M. Jobin, Greenwich, CT (US); Brent A. Ponce, Homewood, AL (US); Edward Rhettson Hobgood, Ridgeland, MS (US); Brian Bowman, Carlsbad, CA (US); Elijah Atkinson, Pflugerville, TX (US)

(73) Assignees: Smith & Nephew, Inc., Memphis, TN (US); Smith & Nephew Orthopaedics AG, Zug (CH); Smith & Nephew Asia Pacific PTE. Limited, Singapore (SG)

(21) Appl. No.: 18/855,708

(22) PCT Filed: Apr. 26, 2023
(86) PCT No.: PCT/US2023/019905

§ 371 Date: Oct. 10, 2024

Related U.S. Application Data
(60) Provisional application No. 63/337,864, filed on May 3, 2022.

(51) Int. Cl. A61F 2/40; A61F 2/30

(52) U.S. Cl. CPC A61F 2/4081; A61F 2002/30878

(57) ABSTRACT

A glenoid implant (100) is disclosed. The glenoid implant including a plurality of bone fixation pegs (130, 140, 150) extending from a back or medial surface thereof. The bone fixation pegs including one or more features in the form of fins (132) and grooves (160) arranged and configured to provide increased fixation to the patient's bone thus minimizing the likelihood of the implant loosening.

### CROSS-REFERENCE TO RELATED APPLICATIONS

This is a non-provisional of, and claims the benefit of the filing date of, pending U.S. provisional patent application No. 63/337,864, filed May 3, 2022, entitled “Glenoid Component,” the entirety of which application is incorporated by reference herein.

### FIELD OF THE DISCLOSURE

The present disclosure relates generally to an orthopedic shoulder implant, and more specifically, to a glenoid implant.

### BACKGROUND OF THE DISCLOSURE

Shoulder replacement surgeries are well known. Shoulder replacement surgery may be used to treat a variety of ailments including, for example, patients suffering from disabling pain due to worn or damaged shoulder joints, which can be caused by, e.g., arthritis or injury.

Shoulder implants are intended to restore the natural kinematics of a patient's shoulder by replacing the patient's humeral head and/or glenoid with similarly shaped orthopedic implants that recreate normal anatomy. With reference to FIG. 1, one known shoulder implant 10 is illustrated. As illustrated, the shoulder implant 10 includes a stem component 20 arranged and configured to be inserted into the patient's humerus bone, a humeral head 30 coupled to the stem component 20, and a glenoid implant 40 arranged and configured to be inserted into the patient's glenoid cavity and to articulate with, relative to, etc. the humeral head 30. That is, as will be readily understood by one of ordinary skill in the art, in use, the humeral head 30 may be coupled to the patient's humerus bone by, for example, resecting the patient's natural humeral head and inserting the stem component 20 into the intramedullary canal of the patient's humerus. Thereafter, the humeral head 30 may be coupled to the stem component 20.

The glenoid implant 40 is coupled within the patient's glenoid cavity. That is, current glenoid implants are intended to sit on a prepared surface of a patient's glenoid bone. The surface is typically prepared by removing any remaining cartilage, reaming a smooth bony surface and by drilling receiving bores for receiving bone fixation pegs extending from the glenoid implant 40. For example, as illustrated, the glenoid implant 40 may include a plurality of bone fixation pegs 42 configured to be inserted into bores formed within the patient's glenoid cavity. The bone fixation pegs 42 extending from a back (e.g., medial surface) of the glenoid implant 40. The bone fixation pegs 42 act as anchoring features to secure the glenoid implant 40 inside the glenoid cavity. In addition, cement is used to affix the glenoid implant 40 within the glenoid cavity.

Generally speaking, the glenoid implant 40 is usually made from a polymer such as ultra-high molecular weight polyethylene (UHMWPE). Thus, generally speaking, glenoid implants may be made completely from polyethylene and affixed to the bone using bone cement and bone fixation pegs. Some glenoid implants may include a rigid base plate made of metal with a polyethylene insert.

One disadvantage of glenoid implants utilizing bone fixation pegs is loosening (e.g., glenoid loosening), which may cause failure of the shoulder implant. For example, these glenoid implants can loosen due to poor fixation to the bone.

Thus, it would be beneficial to provide a glenoid implant having improved fixation. It is with respect to these and other considerations that the present disclosure may be useful.

### SUMMARY OF THE DISCLOSURE

This Summary is provided to introduce a selection of concepts in a simplified form that are further described below in the Detailed Description. This Summary is not intended to identify key features or essential features of the claimed subject matter, nor is it intended as an aid in determining the scope of the claimed subject matter.

A glenoid implant is disclosed. In some examples, the glenoid implant includes a bearing surface arranged and configured to contact a humeral head, a bone contacting surface opposite the bearing surface, and a plurality of bone fixation pegs arranged and configured to be inserted into a plurality of bores formed in a patient's glenoid, the plurality of bone fixation pegs including an outer surface, a plurality of fins extending laterally from the outer surface, and one or more longitudinal grooves formed in the outer surface, the one or more longitudinal grooves configured to enable cement to flow along a length of the peg.

In any preceding or subsequent example, the one or more longitudinal grooves extend through the plurality of fins to form one or more channels, respectively, through the plurality of fins to enable cement to flow between adjacent fins.

In any preceding or subsequent example, the plurality of bone fixation pegs include a primary bone fixation peg and a plurality of secondary bone fixation pegs positioned circumferentially about the primary bone fixation peg.

In any preceding or subsequent example, the primary bone fixation peg includes a first length and a first diameter, the plurality of secondary bone fixation pegs include a second diameter and a second length, the first length being larger than the second length, the first diameter being larger than the second diameter.

In any preceding or subsequent example, the primary bone fixation peg includes three longitudinal grooves formed in the outer surface of the primary bone fixation peg.

In any preceding or subsequent example, the plurality of secondary bone fixation pegs include two diametrically opposed longitudinal grooves formed in the outer surface thereof.

In any preceding or subsequent example, the plurality of fins extending from the primary bone fixation peg include a first fin having a first cross-sectional shape and a second fin having a second cross-sectional shape, the second cross-sectional shape being different from the first cross-sectional shape.

In any preceding or subsequent example, the primary bone fixation peg includes a first end positioned adjacent to the bone contacting surface and a second end opposite the first end, the second fin positioned adjacent to the bone contacting surface.

In any preceding or subsequent example, the primary bone fixation peg includes a plurality of first fins positioned between the second fin and the second end of the primary bone fixation peg.

In any preceding or subsequent example, the first fin includes a front leading surface and a trailing surface, the front leading surface being angled relative to a longitudinal central axis of the primary bone fixation peg.

In any preceding or subsequent example, the trailing surface of the first fin includes an undercut curved surface.

In any preceding or subsequent example, the second fin includes a front leading surface and a trailing surface, wherein the front leading surface and the trailing surface of the second fin are parallel to each other and/or perpendicular to the longitudinal central axis of the primary bone fixation peg.

Examples of the present disclosure provide numerous advantages. For example, by providing one or more longitudinal grooves in the bone fixation pegs, cement is better able to flow along the length of the peg thereby providing improved interdigitation and fixation of the pegs, and thus the glenoid implant, to the patient's bone, reducing the likelihood of glenoid loosening. In addition, by providing one or more longitudinal grooves in the bone fixation pegs, the glenoid implant can be inserted via a manual pushing force thereby eliminating, or at least reducing, the need for mallets and/or hammers. In addition, and/or alternatively, the bone fixation pegs may include fins having different cross-sectional shapes to facilitate easier insertion and/or improved fixation depending on the shape of the fin. In addition, and/or alternatively, by incorporating a curved, arcuate undercut surface in the trailing surface of the fins and/or pegs, the pegs are better able to prevent, or at least minimize, backing out or loosening of the implant.

Further features and advantages of at least some of the examples of the present disclosure, as well as the structure and operation of various examples of the present disclosure, are described in detail below with reference to the accompanying drawings.

### BRIEF DESCRIPTION OF THE DRAWINGS

By way of example, specific examples of the disclosed device will now be described, with reference to the accompanying drawings, in which:

FIG. 1 illustrates an exploded perspective view of a conventional shoulder implant including a stem, a humeral head, and a glenoid;

FIG. 2 illustrates a perspective view of an example of a glenoid implant in accordance with one or more features of the present disclosure, the glenoid implant may be used in the shoulder implant shown in FIG. 1;

FIG. 3 illustrates a bottom view of the glenoid implant shown in FIG. 2;

FIGS. 4A and 4B illustrate various detailed, cross-sectional views of an example of a bone fixation peg in accordance with one or more features of the present disclosure, the bone fixation peg may be used in the glenoid implant shown in FIGS. 2 and 3;

FIG. 5A illustrates a bottom view of an example of an augmented glenoid implant in accordance with one or more features of the present disclosure, the glenoid implant may be used in the shoulder implant shown in FIG. 1;

FIG. 5B illustrates a side view of the augmented glenoid implant shown in FIG. 5A;

FIG. 6A illustrates a bottom view of an example of a standard glenoid implant in accordance with one or more features of the present disclosure, the glenoid implant may be used in the shoulder implant shown in FIG. 1;

FIG. 6B illustrates a side view of the standard glenoid implant shown in FIG. 6A; and

FIG. 7 illustrates a perspective of an example of a glenoid implant including a radio-opaque or radiographic marker in accordance with one or more features of the present disclosure, the glenoid implant may be used in the shoulder implant shown in FIG. 1.

The drawings are not necessarily to scale. The drawings are merely representations, not intended to portray specific parameters of the disclosure. The drawings are intended to depict various examples of the disclosure, and therefore are not considered as limiting in scope. In the drawings, like numbering represents like elements.

### DETAILED DESCRIPTION

Various features or the like of a glenoid implant or component (terms used interchangeably herein without the intent to limit or distinguish) arranged and configured for use in a shoulder implant will now be described more fully herein with reference to the accompanying drawings, in which one or more features of the glenoid implant will be shown and described. It should be appreciated that the various features may be used independently of, or in combination, with each other. It will be appreciated that the glenoid implant as disclosed herein may be embodied in many different forms and may selectively include one or more concepts, features, or functions described herein. As such, the glenoid implant should not be construed as being limited to the specific examples set forth herein. Rather, these examples are provided so that this disclosure will convey certain features of the glenoid implant to those skilled in the art.

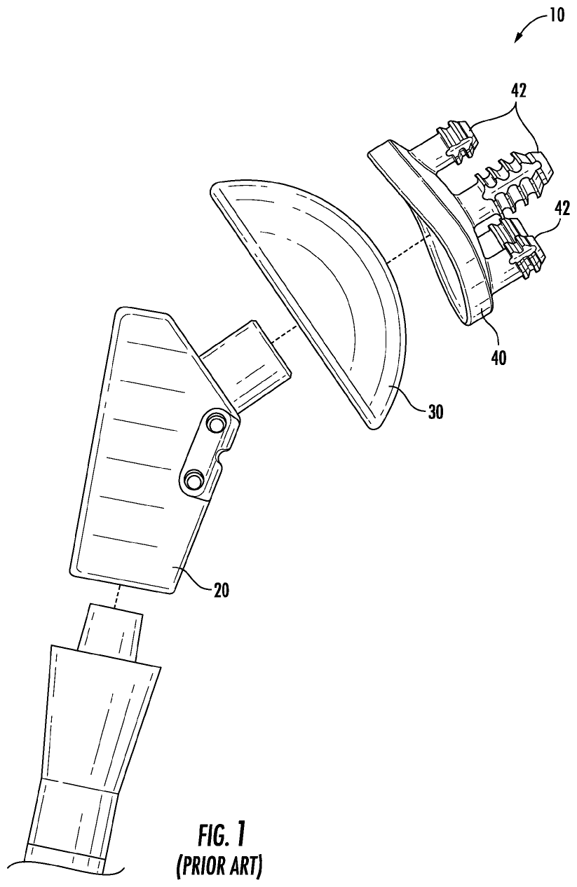

In accordance with one or more features of the present disclosure, a glenoid implant arranged and configured for use in a shoulder implant such as, for example, arranged and configured for use in place of the glenoid implant 40 of the shoulder implant 10 of FIG. 1, is disclosed. As will be described in greater detail herein, in some examples, the glenoid implant includes one or more features arranged and configured to provide increased fixation to the patient's bone.

Generally speaking, and as will be appreciated by one of ordinary skill in the art, in use, the shoulder implant, and more specifically, the glenoid implant such as, for example, glenoid implant 40 of FIG. 1, is implanted by exposing the patient's glenoid cavity. Next a guidewire may be inserted. Using the guidewire, the patient's glenoid may be prepared as needed including, for example, reaming of the glenoid cavity. Thereafter, a plurality of bores, pockets, holes, etc. (terms used interchangeably herein without the intent to distinguish) are formed in the patient's bone corresponding to the number, size, and shape of the bone fixation pegs extending from the selected glenoid implant, as will be described in greater detail herein. The plurality of bores may be formed using, for example, a drill.

With the plurality of bores formed, cement is prepared and injected into each of the bores. In addition, cement may be applied to the bone contacting surface of the glenoid implant. Next, the glenoid implant may be implanted using, for example, a mallet and/or impactor.

One concern with current glenoid implants is that during insertion of the bone fixation pegs into the bores, the cement is compacted into the bore. That is, inserting the bone fixation pegs of the glenoid implant into the bores pushes the cement further into the bore. As such, the cement does not fully interdigitate with and/or cover the bone fixation pegs within the bore, which can lead to loosening of the glenoid implant.

As will be described in greater detail herein, in some examples, an improved glenoid implant including one or more features arranged and configured to provide increased fixation to the patient's bone will be described. In one preferred example, as will be described in greater detail herein, the glenoid implant includes improved bone fixation pegs arranged and configured to enable the cement to flow across and/or around the fins of the bone fixation pegs to enable the cement to better interdigitate with and/or cover the entire length of the bone fixation pegs.

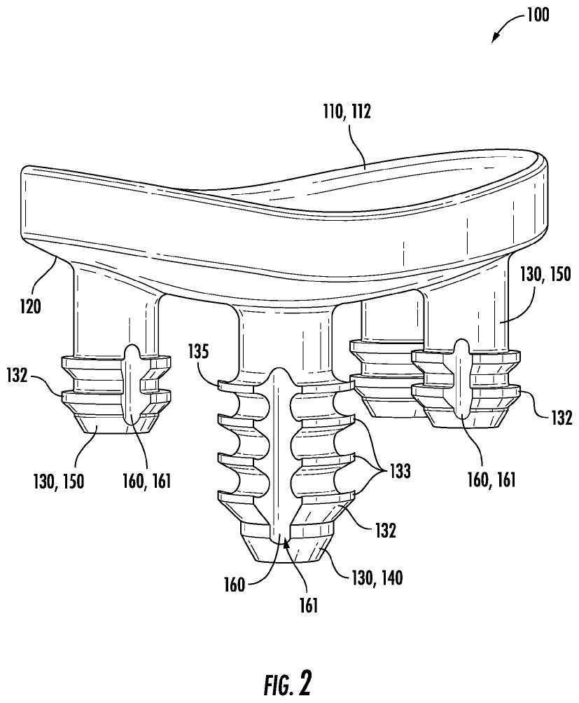

With reference to FIGS. 2-4B, in accordance with one or more features of the present disclosure, a glenoid implant 100 is disclosed. As illustrated, the glenoid implant 100 includes a bearing surface 110. The bearing surface 110 including a concave surface 112 arranged and configured to contact the articulating surface of the humeral head such as, for example, humeral head 30 in FIG. 1. In use, the concave surface 112 can have any suitable shape now known or hereafter developed including, for example, spherical, ellipsoid, hyperboloid, paraboloid, etc. In some examples, the glenoid implant 100 may have an elongated oval shape, however any other suitable shape now known or hereafter developed can be utilized including, for example, a circular shape.

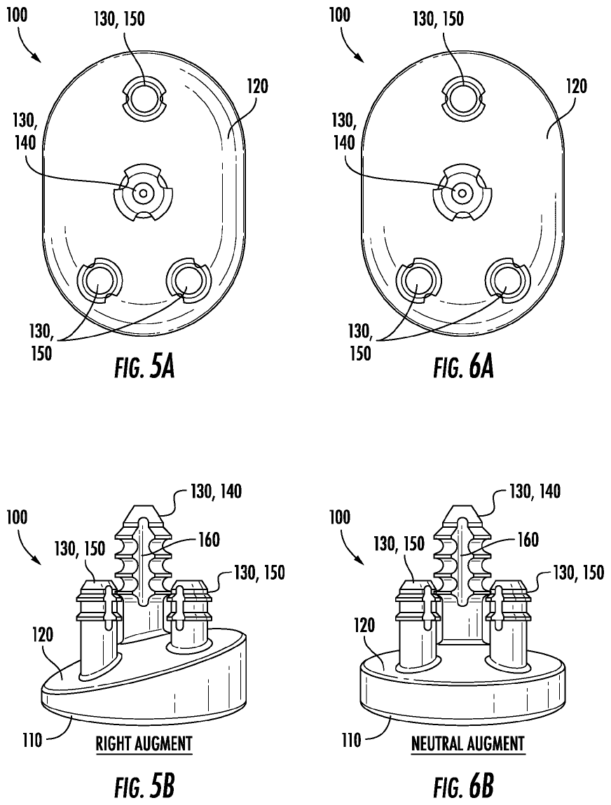

In addition, as illustrated, the glenoid implant 100 includes a bone contacting surface 120 positioned opposite the bearing surface 110 (e.g., concave surface 112). In use, the bone contacting surface 120 can have any suitable shape now known or hereafter developed. For example, with reference to FIGS. 5A-6B, the glenoid implant 100 may be arranged and configured with an angled or augmented bone contacting surface 120 (FIGS. 5A-5B) or a neutral or standard bone contacting surface 120 (FIGS. 6A-6B).

As illustrated in FIGS. 2-4B, the glenoid implant 100 includes a plurality of bone fixation pegs 130 extending from the bone contacting surface 120. In use, the plurality of bone fixation pegs 130 are arranged and configured to be inserted into bores formed in the patient's bone, which, as previously described, include an adhesive such as, for example, cement, positioned therein. As such, in use, the bone fixation pegs 130 couple the glenoid implant 100 to the patient's bone within the patient's glenoid cavity.

As will be appreciated, the present disclosure is directed to an improved bone fixation peg for use on a glenoid implant. Thus, for the sake of brevity, further description of shoulder implants, glenoid implants, etc. is omitted herefrom. In addition, while a specific glenoid implant is shown, it should be appreciated that the improved bone fixation pegs can be used in connection with any glenoid implant now known or hereafter developed. As such, the present disclosure should not be limited to any specific shoulder implant or glenoid implant unless specifically claimed.

As illustrated, in some examples, the glenoid implant 100 may include a total of four bone fixation pegs 130 extending from the bone contacting surface 120 thereof. More preferably, the glenoid implant 100 may include a first primary bone fixation peg 140 and a plurality of secondary bone fixation pegs 150. In some examples, the primary bone fixation peg 140 may be more centrally located on the bone contacting surface 120 of the glenoid implant 100 with the secondary bone fixation pegs 150 positioned about the primary bone fixation peg 140. As illustrated, the primary bone fixation peg 140 may be larger than the secondary bone fixation pegs 150. For example, the primary bone fixation peg 140 may have a longer length (e.g., thus extending farther from the bone contacting surface 120) and a larger diameter than the secondary bone fixation pegs 150. However, it should be appreciated that this is but one example, and that the glenoid implant may include more or less bone fixation pegs including more or less primary bone fixation pegs and secondary bone fixation pegs, and that the secondary bone fixation pegs may be positioned in alternate configurations compared to the primary bone fixation peg.

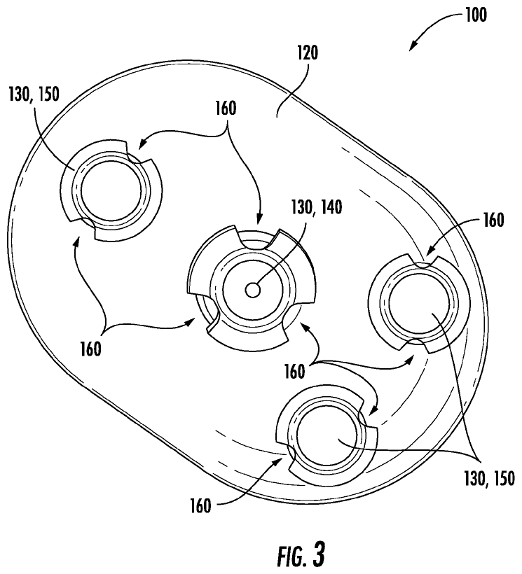

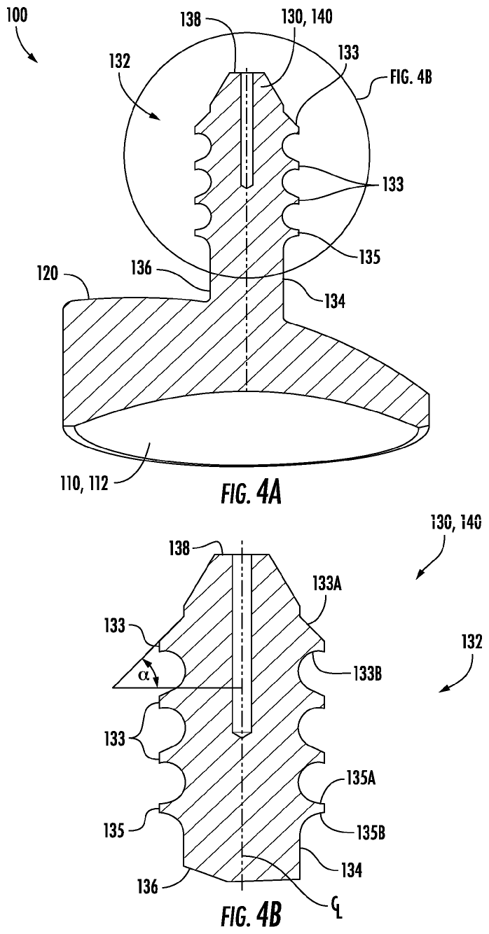

As best illustrated in FIGS. 4A and 4B, the bone fixation pegs 130 including the primary and secondary bone fixation pegs 140, 150 include a generally cylindrical shape with each bone fixation peg including a plurality of ridges, barbs, fins, projections, etc. 132 (terms used interchangeably herein without the intent to distinguish) extending from an outer surface 134 of the bone fixation peg 130. That is, in some examples, each bone fixation peg 130 includes a cylindrical outer body having a first end 136 and a second end 138 along with a plurality of fins 132 extending from the outer surface 134 thereof.

In addition, in accordance with one or more features of the present disclosure, the bone fixation pegs 130 include a longitudinal groove or indentation 160 (terms used interchangeably herein without the intent to distinguish) formed therein. As illustrated, in some examples, the longitudinal groove 160 is formed in the outer surface 134 of the bone fixation peg 130 and extends through one or more of the fins 132. In use, the longitudinal grooves 160 enable cement to flow around and between adjacent fins 132. That is, by forming one or more longitudinal grooves 160 into the outer surface 134 of the bone fixation peg 130 (e.g., by removing material from the outer surface 134 of the bone fixation peg 130 and/or fins 132) one or more channels are formed that enable the cement to flow therethrough thus allowing the cement to obtain better coverage around each fin 132, which creates improved fixation and thus reduces the likelihood of loosening.

That is, as previously described, in use, prior to implantation of the glenoid implant, a patient's bone is pre-drilled with a plurality of bores corresponding to the number and positioning of the bone fixation pegs 130 of the glenoid implant 100. In addition, cement is injected into the bores prior to implantation of the glenoid implant 100. In accordance with one or more features of the present disclosure, by forming one or more longitudinal grooves 160 within the outer surface 134 of the bone fixation pegs 130 and/or through one or more of the fins 132, it has been discovered that the cement positioned within the bores is better able to flow within the bore as the glenoid implant 100 (e.g., bone fixation pegs 130) are being inserted therein. As a result, the cement is able to flow along the length of the longitudinal grooves 160 and into contact with the fins 132 and between the fins 132 at the base of the fins (e.g., adjacent to the outer surface of the bone fixation pegs 130) to provide increased fixation between the bone fixation pegs 130 and the patient's bone via the cement (e.g., tests have found a significant increase in the pull-out strength needed to remove the glenoid implant 100 thus minimizing the risk of post-operative glenoid loosening). This is, in contrast, with conventional glenoid implants utilizing pegs without any longitudinal groove wherein the distal most fin pushes the cement into the bore, thus preventing, or at least minimizing, the cement from interdigitating around and between the proximal fins.

That is, as previously described, the cement is able to flow between the fins 132 at the base of the fins 132 (e.g., adjacent to the outer surface of the bone fixation pegs 130) to provide increased fixation between the bone fixation pegs 130 and the patient's bone via the cement. In some examples, the longitudinal grooves 160 form one or more channels 161 that extend between the fins 132. As illustrated, the fins 132 extend circumferential around the bone fixation peg 130 but for the one or more channels 161 extending therethrough. The fins 132 may be rounded at the base of the channel 161 so that the cement that flows into the channels 161 circumferential around the bone fixation peg 130 to help resist peg pullout even if the apex of the fins bend or deform during attempted peg pullout. The rounded base channels may also reduce cement fragmentation during attempted pullout because the forces are distributed evenly to the rounded geometry of the cement in these channels.

In addition, by incorporating one or more longitudinal grooves 160 in the outer surface 134 of the bone fixation pegs 130 and/or through one or more of the fins 132, it has been discovered that the glenoid implant 100 can be inserted with a manual pushing force by, for example, the surgeon. That is, the force required to insert the glenoid implant 100 has been found to be reduced thus allowing surgeons to manually insert the glenoid implant 100.

In some examples, the bone fixation pegs 130 may include any number and/or configuration of longitudinal grooves 160 formed therein arranged and configured to enable cement to flow therethrough. For example, as illustrated in FIGS. 2 and 3, each of the smaller, secondary bone fixation pegs 150 may include two longitudinal grooves 160 formed in an outer surface thereof. As illustrated, in some examples, the longitudinal groves 160 may be positioned on diametric sides of the longitudinal central axis C_{L} of the secondary bone fixation pegs 150. Alternatively, as illustrated in FIGS. 2 and 3, the larger primary bone fixation peg 140 may include three longitudinal grooves 160 formed in an outer surface thereof. These are but two examples and it is envisioned that the pegs may include any number and arrangement of grooves formed therein including, one, four, five, or more. Thus, for example, the primary and secondary bone fixation pegs 140, 150 may include equal number and/or configurations of grooves, or they may be different.

In addition, and/or alternatively, in accordance with one or more features of the present disclosure that may be used in combination with or separately from the longitudinal grooves 160, the bone fixation pegs 130 may include differently shaped fins 132 depending on their location along the bone fixation pegs 130 (e.g., differently shaped fins may be used along a longitudinal length of the longitudinal central axis C_{L} of the bone fixation peg 130). That is, for example, the bone fixation pegs 130 may include first and second shapes, configurations, forms, etc. of fins (e.g., first and second fins wherein the cross-sectional shape of the first fin type is different from the cross-sectional shape of the second fin type). That is, as best illustrated in FIGS. 2, 4A, and 4B, the bone fixation pegs 130, and more specifically, the primary bone fixation peg 140, may include a plurality of first fins 133 and a second fin 135 having a different cross-sectional shape as compared to the first fins 133. In use, while first and second fins 133, 135 will be described and shown in connection with the primary bone fixation peg 140, it is envisioned that the secondary bone fixation pegs 150 may also include first and second fin types.

As best illustrated in FIG. 4B, the first fin 133 may include a front leading surface 133A and a trailing surface 133B. In some examples, the front leading surface 133A may be angled relative to a longitudinal central axis C_{L} of the primary bone fixation peg 140 by an angle α. In some examples, angle α may be approximately 45 degrees. In some examples, the trailing surface 133B includes an undercut curved surface. As such, the first fins 133 may give the primary bone fixation peg 140 the appearance of a Christmas tree, with an angled leading surface and a curved, arcuate undercut to facilitate improved fixation into the bone. Thus arranged, in use, the front leading surface 133A is arranged and configured to enable easier insertion of the glenoid implant 100 (e.g., configured to enable easier insertion of the primary bone fixation peg 140 into the bore formed in the patient's glenoid cavity). The trailing surface 133B is arranged and configured to enable improved fixation by enabling improved purchase into the prepared bone.

Meanwhile, the second fin 135, which is positioned closer to and/or adjacent to the bone contacting surface 120 of the glenoid implant 100 includes a front leading surface 135A and a trailing surface 135B wherein the front leading surface 135A and the trailing surface 135B are substantially parallel to each other and/or substantially perpendicular to the longitudinal central axis C_{L} of the primary bone fixation peg 140. Thus arranged, the second fin 135 positioned adjacent to the bone contacting surface 120 of the glenoid implant 100 is arranged and configured to facilitate improved fixation (e.g., second fin type provides increased resistance to pulling out). As illustrated, in one preferred example, the primary bone fixation peg 140 includes a single fin having the second fin type, however it is envisioned that additional fins may include the second fin type.

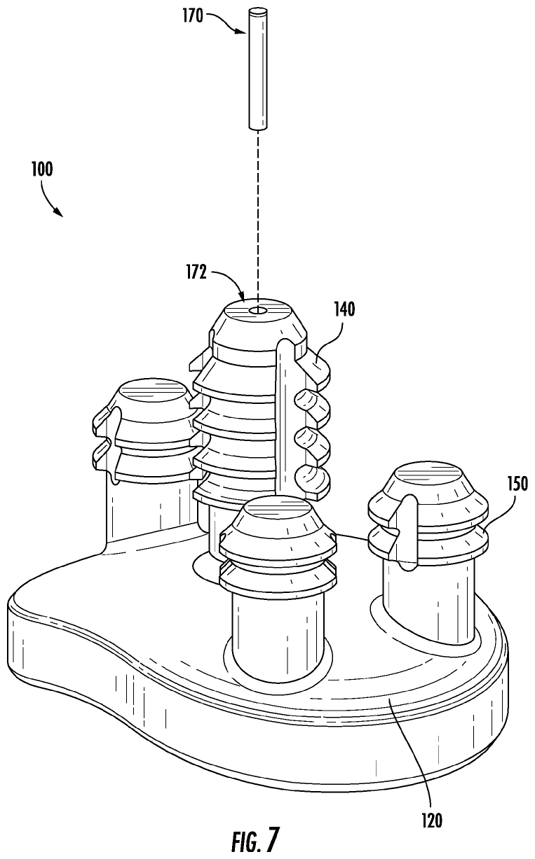

In some examples, as illustrated in FIG. 7, one or more of the bone fixation pegs 130, shown as primary bone fixation peg 140, may include a borehole 172 arranged and configured to receive a radio-opaque or radiographic marker 170.

In addition, in some examples, one or more of the bone fixation pegs 130 can include a lumen arranged and configured to enable cement to be injected therein. The bone fixation pegs 130 can also include pores, slits, or holes formed in the outer surface thereof and positioned, for example, adjacent to each fin to enable the injected cement to flow out.

In some examples, the glenoid implant 100 is manufactured of a plastic material such as, for example, ultra-high molecular weight polyethylene (UHMWPE), although other suitable materials such as, for example, other suitable plastic or metals can be utilized.

The glenoid implant 100 can be manufactured by any suitable process now known or hereafter developed. For example, the glenoid implant 100 can be manufactured via CNC machining, 3D-printing, etc.

In some examples, the bone contacting surface 120 may be roughened in any suitable manner known in the art in order to facilitate bone in-growth. In some examples, if the implant is manufactured from a metal, the bone contacting surface 120 could include a grit-blasted, titanium plasma sprayed. Hydroxylapatite (HA) coating or other on/in-growth coatings used in joint replacement may be applied to the bone contacting surface.

While the present disclosure refers to certain examples, numerous modifications, alterations, and changes to the described examples are possible without departing from the sphere and scope of the present disclosure, as defined in the appended claim(s). Accordingly, it is intended that the present disclosure not be limited to the described examples, but that it has the full scope defined by the language of the following claims, and equivalents thereof. The discussion of any example is meant only to be explanatory and is not intended to suggest that the scope of the disclosure, including the claims, is limited to these examples. In other words, while illustrative examples of the disclosure have been described in detail herein, it is to be understood that the inventive concepts may be otherwise variously embodied and employed, and that the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more examples or configurations for the purpose of streamlining the disclosure. However, it should be understood that various features of the certain examples or configurations of the disclosure may be combined in alternate examples, or configurations. Any example or feature of any section, portion, or any other component shown or particularly described in relation to various examples of similar sections, portions, or components herein may be interchangeably applied to any other similar example or feature shown or described herein. Additionally, components with the same name may be the same or different, and one of ordinary skill in the art would understand each component could be modified in a similar fashion or substituted to perform the same function.

Moreover, the following claims are hereby incorporated into this Detailed Description by this reference, with each claim standing on its own as a separate example of the present disclosure.

As used herein, an element or step recited in the singular and proceeded with the word “a” or “an” should be understood as not excluding plural elements or steps, unless such exclusion is explicitly recited. Furthermore, references to “one example” of the present disclosure are not intended to be interpreted as excluding the existence of additional examples that also incorporate the recited features.

The phrases “at least one.” “one or more.” and “and/or.” as used herein, are open-ended expressions that are both conjunctive and disjunctive in operation. The terms “a” (or “an”), “one or more” and “at least one” can be used interchangeably herein. All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., engaged, attached, coupled, connected, and joined) are to be construed broadly and may include intermediate members between a collection of elements and relative to movement between elements unless otherwise indicated. As such, connection references do not necessarily infer that two elements are directly connected and in fixed relation to each other. All rotational references describe relative movement between the various elements. Identification references (e.g., primary, secondary, first, second, third, fourth, etc.) are not intended to connote importance or priority but are used to distinguish one feature from another. The drawings are for purposes of illustration only and the dimensions, positions, order and relative to sizes reflected in the drawings attached hereto may vary.

1. A glenoid implant comprising:
a bearing surface arranged and configured to contact a humeral head;
a bone contacting surface opposite the bearing surface; and
a plurality of bone fixation pegs arranged and configured to be inserted into a plurality of bores formed in a patient's glenoid, the plurality of bone fixation pegs including an outer surface, a plurality of fins extending laterally from the outer surface, and one or more longitudinal grooves formed in the outer surface, the one or more longitudinal grooves configured to enable cement to flow along a length of the peg;
wherein the plurality of fins extending from the primary bone fixation peg include a first fin having a first cross-sectional shape and a second fin having a second cross-sectional shape, the second cross-sectional shape being different from the first cross-sectional shape.

2. The glenoid implant of claim 1, wherein the one or more longitudinal grooves extend through the plurality of fins to form one or more channels, respectively, through the plurality of fins to enable cement to fiow between adjacent fins.

3. The glenoid implant of claim 1, wherein the plurality of bone fixation pegs include a primary bone fixation peg and a plurality of secondary bone fixation pegs positioned circumferentially about the primary bone fixation peg.

4. The glenoid implant of claim 3, wherein the primary bone fixation peg includes a first length and a first diameter, the plurality of secondary bone fixation pegs include a second diameter and a second length, the first length being larger than the second length, the first diameter being larger than the second diameter.

5. The glenoid implant of claim 4, wherein the primary bone fixation peg includes three longitudinal grooves formed in the outer surface of the primary bone fixation peg.

6. The glenoid implant of claim 5, wherein the plurality of secondary bone fixation pegs include two diametrically opposed longitudinal grooves formed in the outer surface thereof.

7. (canceled)

8. The glenoid implant of claim 1, wherein the primary bone fixation peg includes a first end positioned adjacent to the bone contacting surface and a second end opposite the first end, the second fin positioned adjacent to the bone contacting surface.

9. The glenoid implant of claim 8, wherein the primary bone fixation peg includes a plurality of first fins positioned between the second fin and the second end of the primary bone fixation peg.

10. The glenoid implant of claim 8, wherein the first fin includes a front leading surface and a trailing surface, the front leading surface being angled relative to a longitudinal central axis of the primary bone fixation peg.

11. The glenoid implant of claim 10, wherein the trailing surface of the first fin includes an undercut curved surface.

12. The glenoid implant of claim 10, wherein the front leading surface is angled relative to the longitudinal central axis of the primary bone fixation peg by an angle α, angle α being approximately 45 degrees.

13. The glenoid implant of claim 10, wherein the second fin includes a front leading surface and a trailing surface, wherein the front leading surface and the trailing surface of the second fin are parallel to each other.

14. The glenoid implant of claim 13, wherein the front leading surface and the trailing surface of the second fin are perpendicular to the longitudinal central axis of the primary bone fixation peg.

15. The glenoid implant of claim 1, wherein the primary bone fixation peg includes a borehole arranged and configured to receive a radio-opaque or radiographic marker.

16. A glenoid implant comprising:
a bearing surface arranged and configured to contact a humeral head;
a bone contacting surface opposite the bearing surface; and
a plurality of bone fixation pegs arranged and configured to be inserted into a plurality of bores formed in a patient's glenoid, the plurality of bone fixation pegs including an outer surface and a plurality of fins extending laterally from the outer surface;
wherein the plurality of fins extending from the primary bone fixation peg include a first fin having a first cross-sectional shape and a second fin having a second cross-sectional shape, the second cross-sectional shape being different from the first cross-sectional shape.

17. The glenoid implant of claim 16, wherein the plurality of bone fixation pegs include one or more longitudinal grooves formed in the outer surface, the one or more longitudinal grooves extending through the plurality of fins to form one or more channels, respectively, to enable cement to flow along a length of the peg

18. The glenoid implant of claim 16, wherein the plurality of bone fixation pegs include a primary bone fixation peg and a plurality of secondary bone fixation pegs positioned circumferentially about the primary bone fixation peg;
wherein the primary bone fixation peg includes a first length and a first diameter, the plurality of secondary bone fixation pegs include a second diameter and a second length, the first length being larger than the second length, the first diameter being larger than the second diameter.

19. The glenoid implant of claim 16, wherein the primary bone fixation peg includes a first end positioned adjacent to the bone contacting surface and a second end opposite the first end, the second fin positioned adjacent to the bone contacting surface.

20. The glenoid implant of claim 19, wherein the primary bone fixation peg includes a plurality of first fins positioned between the second fin and the second end of the primary bone fixation peg.

21. The glenoid implant of claim 19, wherein the first fin includes a front leading surface and a trailing surface, the front leading surface being angled relative to a longitudinal central axis of the primary bone fixation peg.

22. The glenoid implant of claim 21, wherein the trailing surface of the first fin includes an undercut curved surface.

23. The glenoid implant of claim 21, wherein the front leading surface is angled relative to the longitudinal central axis of the primary bone fixation peg by an angle α, angle α being approximately 45 degrees.

24. The glenoid implant of claim 21, wherein the second fin includes a front leading surface and a trailing surface, wherein the front leading surface and the trailing surface of the second fin are parallel to each other.

25. The glenoid implant of claim 24, wherein the front leading surface and the trailing surface of the second fin are perpendicular to the longitudinal central axis of the primary bone fixation peg.
